(19) United States
(12) Patent Application Publication
(10) Pub. No.: US 2007/0104769 A1
(43) Pub. Date: May 10, 2007

(54) Bioabsorbable hemostatic gauze

(75) Inventors: Vicky Feng, San Jose, CA (US); David H. Hsu, Diamond Bar, CA (US); Yan Yin, Fremont, CA (US)

Correspondence Address: MICHAELSON & ASSOCIATES, P.O. BOX 8489, RED BANK, NJ, 07701, US

(73) Assignee: LifeScience PLUS, Inc.

(21) Appl. No.: 11/591,154

(22) Filed: Nov. 1, 2006

Related U.S. Application Data
(60) Provisional application No. 60/733,322, filed on Nov. 4, 2005.
(60) Provisional application No. 60/846,314, filed on Sep. 21, 2006.

(51) Int. Cl. A61K 31/717; C08B 11/20; A61L 15/00

(52) U.S. Cl. 424/445; 514/57; 536/89

(57) ABSTRACT

Bioabsorbable, water-soluble hemostatic cellulose based gauze matrix structures are described, including one or more species of chitosan, etherized cellulose, nonionic surfactant, water-soluble polysaccharide hydrocolloid and/or gum. Approximately 85% to 95% deacetylated decrystallized chitosan, present in an amount from about 2% to about 15% by weight, is found to be particularly advantageous. Favorable properties are found related to rapid stoppage of bleeding and bioabsorbability, among other properties.

### CLAIM TO PRIORITY

This application claims the benefit of our co-pending provisional patent application entitled “Chitosan Modified Etherized Soluble, Absorbable Hemostat,” filed Nov. 4, 2005 and assigned Ser. No. 60/733,322, the entire contents of which is incorporated herein by reference for all purposes. This application also claims the benefit of our co-pending provisional patent application entitled “Bioabsorbable Haemostatic Gauze,” filed Sep. 21, 2006 and assigned Ser. No. 60/846,314, the entire contents of which is incorporated herein by reference for all purposes.

### BACKGROUND OF THE DISCLOSURE

1. Field of the Invention

The invention relates generally to hemostatic compositions and gauze matrix structures containing hemostatic compositions, and more particularly, to bioabsorbable, water soluble cellulose based compositions for arresting bleeding.

2. Description of the Prior Art

A major cause of death among accident victims and military personnel wounded in action is hemorrhage. The use of first aid to control topical bleeding is thus of critical importance.

Cellulose based materials, such as cellulose gauze made from cotton or regenerated cellulose fiber, regenerated cellulose sponge, other cellulose fibers and the like have been utilized to absorb body fluids and blood during surgery. A major disadvantage in the use of such products in contact with tender or sensitive areas of the body, such as the eye, abrasions, incisions and the like, is the typically stiff, harsh and/or scratchy nature of cellulose sponges and fibers. These properties of cellulose can result in irritation, may cause a rupture of the skin or membrane and result in infection in the wound area. When the cellulose material is cut to various sizes, perhaps by a paramedic or first-responder under emergency, time-critical conditions, the sharp edges of the cut surfaces can cause irritation. Also, loose fiber fragments are typically formed along the cut surfaces. Thus, when this cut cellulose material is used in eye areas, open wounds and/or surgery, the cut surfaces can cause irritation. In addition, loose fiber fragments may further irritate the skin or membrane and may serve as a source of infection.

U.S. Pat. No. 4,543,410 describes absorbent cellulose structures which overcome some of the disadvantages of the prior products but still have some important disadvantages. For example, when loose fibers from these structures enter a wound, it may not be feasible to detect and remove all such fibers by visual inspection. Since water-insoluble cellulose material is not absorbed by the body, it may serve as a source of contamination or infection and complicate the wound healing process and hinder the prompt recovery of the patient.

For treating external hemorrhage, cotton gauze pads with the capability of absorbing about 250 mL (milliLiter) of blood are the main dressings currently in use by the military and by civilian trauma units. These pads are dressings typically used passively, that is, such dressing do not typically initiate or accelerate blood clotting.

A hemostatic pressure bandage containing fibrin glue formed by combining bovine fibrinogen and thrombin was proposed by Larson, M. J., et al, Arch. Surg. 130:420-422 (1995), the purpose of which is to control injured arteries in a swine model.

U.S. Pat. No. 6,056,970 discloses solid, fibrous bioabsorbable hemostatic compositions containing a bioabsorbable polymer, and hemostatic compounds such as thrombin or fibrinogen. One disadvantage of using fibrin glue as well as collagen or other materials derived from animals or animal products is the inherent risk of transmitting disease or other contaminants by means of the hemostatic composition. That is, the blood or other substances serving as the source of one or more of the components of the hemostatic composition may include disease-bearing or other substances harmful to the patent for whom the hemostatic composition is intended, which may slip through any purification procedure or add to the cost of the product by the necessity of exceptionally thorough purification.

Another major disadvantage of some products in the prior art is that, to effectively stop bleeding, the components must be kept separated during storage and transport and combined at the time of use. The thrombin component, for example, degrades at high temperature and typically must be maintained at a temperature of 30 deg. C. or below—not always convenient in first aid kits intended for use in hot environments or under circumstances in which cooling during storage and transport is unavailable.

Chitosan is a partially or fully deacetylated form of chitin, a naturally occurring polysaccharide. Like chitin, chitosan is a generic term for a group of polymers of acetylglucosamine, but with a degree of deacetylation of between about 50 and 90 percent. Chitosan is a polysaccharide containing primary amine groups and includes a distribution of the β-(1-4)-linked D-glucosamine (deacetylated unit) and N-acetyl-D-glucosamine (acetylated unit). Chitosan is structurally similar to cellulose, with a difference being that the C-2 hydroxyl group in cellulose is substituted with a primary amine group in chitosan. The large number of free amine groups (having pKₐ about 6.3) makes chitosan a polymeric weak base. Both chitin and chitosan are insoluble in water, dilute aqueous bases, and most organic solvents. However, unlike chitin, chitosan is soluble in dilute aqueous acids, usually carboxylic acids, as the chitosonium salt. Solubility in dilute aqueous acid is therefore a simple way to distinguish chitin from chitosan.

Chitosan and its derivatives are useful in making surgical dressings and sutures, ocular bandages and lenses. Chitosan is capable of forming water-soluble salts with many organic and inorganic acids. Such salts are typically biologically compatible with skin, hair, and most living tissues. Since some chitosan salts can promote rapid healing in damaged tissue, such chitosonium derivatives can be useful in biomedical applications. The tissue compatibility and healing acceleration properties of these chitosan salts are also shared by many covalent chitosan derivatives, covalent chitin derivatives, chitosan, and chitin.

However, chitosan derivatives are typically highly crystalline polymers and are difficult to prepare. U.S. Pat. No. 4,929,722 discloses a decrystallization process which can render chitosan into an amorphous structure swollen with diluent. This form of chitosan is typically more amenable to the formation of derivative compounds.

U.S. Pat. No. 5,800,372 proposes microfibrillar collagen and a superabsorbent polymer combined in a hemostatic bandage which both absorbs blood and induces clotting. However, its disadvantages include relatively slow stoppage of bleeding, poor controllability in terms of bioabsorbency and the risk of transferring disease from the collagen donor to the patient.

U.S. Pat. No. 5,047,244 describes a mucoadhesive carrier which releases therapeutic agent in a controlled manner via the mucosal tissue. It comprises an anhydrous but hydratable polymer matrix and amorphous fumed silica. An optional water-insoluble film can be added to provide a non-adhering surface. In WO 91/06270, the same authors disclose a trilaminate film for prolonging the delivery of an active ingredient in the oral cavity.

U.S. Pat. No. 4,876,092 discloses a sheet-shaped adhesive preparation. It comprises an adhesive layer which contains water-soluble and water-insoluble polymers and a water-insoluble carrier. It can adhere to the oral mucosa thereby releasing an active agent to the oral cavity. However, these devices are not completely bioabsorbable and, thus, will stay in the oral cavity after the treatment is completed, leaving the patient with a certain discomfort, resulting mainly from the support layer which leaves an insoluble residue in the mouth.

In order to reduce the adverse feeling in the oral cavity caused by the rigidity and inflexibility of the support layer, a number of attempts have been made introducing soft film supports. EP-0-200-508-B1 and EP-0-381-194-B1 disclose the use of polyethylene films, polyvinyl acetate, ethylene-vinyl acetate copolymers, metal foils, laminates of cloth or paper and a plastic film, and similar materials as soft film supports, whereby synthetic resin-like polyethylene, vinyl acetate homopolymers, and ethylene-vinyl acetate are the preferred materials. CA-1-263-312 discloses the use of polyolefines such as polyethylene, polypropylene, polyesters, PVC, and non-woven fabrics as soft support materials.

However, these devices still leave the patient with a considerable amount of residue due to the water-insoluble nature of the support film. This causes a feeling of discomfort. One approach to overcoming this problem has been to develop a completely degradable mucoadhesive film or a film completely dissolvable in saliva. Fuchs and Hilmann (DE-24-49-865.5) describe a homogeneous, water-soluble cellulose derivatives, such as hydroxyethyl cellulose, hydroxypropyl cellulose, or methyl hydroxypropyl cellulose, as film forming agents.

Both DE-36-30-603 and EP-0-219-762 disclose the use of swellable polymers (such as gelatin or corn starch) as film forming agents, which disintegrate slowly upon application to the oral cavity, thereby releasing an active ingredient incorporated in the film. The same polymers can also be used to prepare films which are intended for dental cleansing, as described in EP-0-452-446-B1. Because of the initial rigidity and delayed softening of these preparations, they still create an adverse feeling in the mouth. Thus, there remains a need in the art for a composition for use in the oral cavity which reduces or avoids feelings of discomfort in the patient's mouth.

U.S. Pat. No. 6,177,096 discloses methods and compositions for avoiding an adverse feeling by incorporating water-soluble polymers with one or more plasticizers or surfactants, one or more polyalcohols, and a pharmaceutically or cosmetically active ingredient which is intended for application to the oral mucosa with instant wettability. Bioabsorbability is an improvement over many previous non-bioabsorbable dressings. However, one disadvantage relates to the limited liquid absorbency, slow blood stopping ability, and poor controllability in terms of its body absorbency.

The use of polysaccharides as wound dressings and wound treatment compositions has also been investigated. Alginate gels, films, fibers and/or fabrics have been proposed as wound dressings. The solubility of the alginate can be varied depending on the ratio of sodium alginate (soluble) to calcium alginate (insoluble) in the compositions.

EP-A-0227553 describes a sodium/calcium alginate sponge for use as a hemostatic dressing. The sponge is formed by mixing an aqueous solution of sodium alginate with a solution of calcium chloride in an inert atmosphere. The mixture is then freeze-dried. It is reported that the resulting alginate sponges are either too soluble (at high sodium contents), or too brittle (at high calcium contents) to be advantageous for use as wound dressings.

Other polysaccharides have been proposed for use as, or in, wound dressing materials, which include glucosaminoglycans, such as hyaluronic acid and its derivatives and heparin, and other naturally occurring polysaccharides, such as chitin. The use of naturally occurring polysaccharide gums to form wound dressing gels has also been proposed. In particular, WO-A-9106323 and WO-A-9306802 suggest the use of xanthan or guar gums as gelling agents in the preparation of wound dressing gels. U.S. Pat. No. 4,994,277 describes the use of certain aqueous gels containing xanthan in surgery for the reduction of tissue adhesions. These gels may also contain a galactomannan such as guar gum to increase viscosity, or gelatin. U.S. Pat. No. 4,341,207 describes a multi-layer decubitus ulcer dressing including a wound-contacting layer comprising a mixture of water soluble or swellable hydrocolloids such as guar gum and other binders for the hydrocolloids.

The use of a mixture of these two polysaccharide types may result in a material having a controllable solubility that can be adjusted for each wound dressing application. U.S. Pat. No. 6,309,661 describes freeze-dried solid, bioabsorbable dressings made with a mixture of xanthan gum and at least one galactomannan, such as guar gum or locust bean gum, noting that the weight ratio of xanthan to total galactomannans should be in the range of 10:90 to 90:10. More preferably, the ratio is in the range 25:75 to 75:25. The bioabsorbability is an improvement over many previous non-bioabsorbable dressing and can typically control water solubility better than other conventional dressings. The disadvantages include the relatively poor performance in rapidly stopping bleeding, in speed of healing, in liquid absorbing capacity, and in speed of liquid absorption.

Thus, a need exists in the art for hemostatic compositions and products having improved performance characteristics in one or more of the following: speed and/or controllability of bioabsorption, reduced risk of infection and/or irritation, reduced side effects, hypoallergenic, speed of arresting bleeding and/or promoting healing, high liquid absorbency, rapid liquid absorption, controllable consistency and durability, among others.

### SUMMARY OF THE INVENTION

Some embodiments of this invention relate generally to bioabsorbable, water-soluble, hemostatic cellulose based gauze matrix structures, hemostatic compositions useful in cooperation with gauze structures, methods of fabrication and use, as well as products useful for wound treatment. Such gauze matrix structures pursuant to some embodiments of the present invention are capable of one or more of the following when judged in comparison with many other treatments and treatment products currently in use: rapidly arresting bleeding from a wound, and/or reducing or minimizing the risk of infection, and/or enhancing the speed of healing and/or reducing harmful side-effects. The hemostatic cellulose based gauze pursuant to some embodiments of the present invention typically includes some or all of the following: one or more species of etherized cellulose, chitosan, one or more species of water-soluble polysaccharide hydrocolloid, and one or more species of nonionic surfactant. Upon application of the structure(s) to the body, the aqueous body fluids typically swell the fiber, protruding fibers and fibrils, and thereby facilitate sharp edges being dissolved or smoothed out. Thus, irritation is reduced or substantially eliminated.

The gauze matrix structures pursuant to some embodiments of the present invention generally exhibit excellent hemostatic properties. The inclusion of uniformly dispersed polysaccharide hydrocolloids in some embodiments of the present invention typically enhances hemostatic efficacy. The use of polysaccharide gums and nonionic surfactant species in some embodiments of the present invention typically enhances the controlled solubility properties of the gauze.

Some embodiments of the present invention relate to the preparation of suitable bioabsorbable, water-soluble, hemostatic cellulose based gauze matrix structures with advantageously short bioabsorption times, advantageous capabilities for rapidly arresting bleeding from a wound, reducing or minimizing the risk of infection, enhancing the speed of healing, and reducing or eliminating side-effects.

It is an objective of some embodiments of the present invention to provide improved solid bioabsorbable hemostatic materials as typically used for wound dressings. The desired properties for the improved materials pursuant to some embodiments of the present invention include one or more of the following: rapid stoppage of bleeding, rapid healing, controllable solubility in body fluids, short bioabsorption time in the body, reduced side effects, hypoallergic, low risk of infection, high liquid absorbency, rapid liquid absorbency, durable and controllable consistency, biodegradability, and low cost.

It is an objective of some embodiments of the present invention to provide improved solid bioabsorbable hemostatic materials that can be promptly absorbed by the body, so the materials need not be removed from the body after surgery or other treatment. This property, among others, can simplify surgical procedures and reduce the pain and suffering during post-operative recovery.

It is an objective of some embodiments of the present invention to produce solid bioabsorbable hemostatic materials by combining some or all the benefits of using the following components: (1) water soluble bioabsorbable cellulose polymers which provide rapid stoppage of bleeding, rapid healing, with rapid bioabsorption, high liquid absorbency and biodegradability; (2) Fully deacetylated and decrystallized chitosans which impart reduced stiffness, improved wet/dry strength ratio, and reduced Tinting and sloughing; (3) surface active agent(s) which enhance rapid wettability and can leave a pleasant feeling in the mouth when it is used as an oral hemostatic dressing (also, in some cases, acting synergistically with chitosan to soften the cellulose, reduce the Tinting and sloughing); (4) polysaccharide gums which provide controllable solubility in body fluids and can function synergistically with etherized cellulose in its bioabsorbability.

Accordingly, some embodiments of the present invention provide a solid bioabsorbable material for use as an effective wound dressing by activating the coagulation factor(s).

It is a further objective of some embodiments of the present invention to provide a method of making etherized celluloses having desirable properties.

It is a further objective of some embodiments of the present invention to provide a method of making solid bioabsorbable materials having one or more of the desired characteristics enumerated above.

### DETAILED DESCRIPTION

Some embodiments of the present invention relate to one or more of the following:

A composition of bioabsorbable, water-soluble, hemostatic gauze matrix with short bioabsorption time, containing one or more etherized celluloses present in the amount of about 55% to about 95% by weight, chitosan in the range from about 0.5% to about 15% by weight, one or more water-soluble polysaccharide gums in the range from about 5% to about 50% by weight, one or more nonionic surfactants in the range from about 0.1% to about 5% by weight, and acetic acid (advantageously reagent grade) in the range from about 0.01% to about 10%. The etherized cellulose is typically selected from the group consisting of hydroxy propyl cellulose, methyl hydroxy propyl cellulose, methyl hydroxy ethyl cellulose. A more advantageous percent by weight of the compositions is from about 65% to about 85% of one or more etherized cellulose, from about 1% to about 5% of chitosan, from about 15% to about 25% of one or more water-soluble polysaccharide hydrocolloids and from about 0.2% to about 2.0% one or more surfactants.

The etherized cellulose advantageously used in some embodiments of the composition is selected from the group consisting of hydroxy propyl cellulose, methyl hydroxy propyl cellulose, and methyl hydroxy ethyl cellulose. The composition can include individual etherized polymer, combination of any two, or combination of all three in various ratios. Our research indicates that the most advantageous combination is hydroxy propyl cellulose, methyl hydroxy propyl cellulose, and methyl hydroxy ethyl cellulose in the ratio of approximately 1:2:1.5

The etherized cellulose pursuant to some embodiments of the present invention is advantageously selected from the group consisting of hydroxy propyl cellulose with DS (Degree of Substitution) in the range from about 0.3 to about 2.5, methyl hydroxy propyl cellulose with DS in the range from about 0.6 to about 2.8, and methyl hydroxy ethyl cellulose with DS in the range from about 0.5 to about 2.6. Particularly advantageous etherized celluloses are selected from the group consisting of hydroxy propyl cellulose with DS in the range from about 0.8 to about 2.0, methyl hydroxy propyl cellulose with DS in the range from about 1.2 to about 2.5, and methyl hydroxy ethyl cellulose with DS in the range from about 1.0 to about 2.2.

The etherized celluloses prepared pursuant to some embodiments of the present invention typically possess one or more advantageous properties, including one or more of the following: rapid stoppage of bleeding, rapid healing, short bioabsorption time, high liquid absorbency and biodegradability. The short bioabsorption time is among the particularly advantageous features of some embodiments of the present invention. The hemostatic dressing with the shortest bioabsorption time currently on the market has a bioabsorption time of about 48 hours. However, gauze matrix structures pursuant to some embodiments of the present invention can be completely dissolved in the body in as short as 2 hours. This short bioabsorption time means the body will have decreased resistance to the dressing material, which is essentially a foreign material left in the body after surgery. Hence, such embodiments provide a high standard of safety and reduced risk of infection.

Chitosan is a high molecular weight linear carbohydrate typically comprising acetylated and deacetylated units. Chitosan is a deacylated derivative of chitin. Chitin is a glucosamine polysaccharide structurally similar to cellulose. Chitin is typically produced in commercial quantities from the shells of crustaceans. Chitin is insoluble in most common solvents. However, chitosan is soluble in acidified water due to the presence of basic amino groups. Depending on the source and degree of deacetylation, chitosans can vary in molecular weight and in free amine content. In sufficiently acidic environments the amino groups become protonated and chitosan behaves as a cationic polyelectrolyte. Chitosan has been used as an effective dry strength additive for paper among other uses.

The chitosan used in some compositions pursuant to some embodiments of the present invention is selected from the group consisting of approximately 85% to 90% deacetylated decrystallized chitosan. Especially advantageous is a ratio of etherized cellulose to deacetylated decrystallized chitosan in the range of approximately 10:1 to approximately 15:1.

When 85% to 90% deacetylated decrystallized chitosan is used, in order to achieve substantially complete water solubility, the pH of the fibrous pulp in the preparation stage should be adjusted to lie in the range of about 4.5-6.0, typically adjusted with reagent grade acetic acid (typically 84% weight to weight, w/w). When the water-soluble deacetylated decrystallized chitosan is used in combination with the suggested nonionic surfactants in the suggested composition, the gauze matrix is significantly softened, with an improved wet/dry strength ratio, and reduced linting and sloughing. These properties are quite advantageous in reducing or substantially eliminating the possibility of contamination in the wound area. The percentage of the 85% to 90% deacetylated decrystallized chitisen used in the formulation also affects the bioabsorption time. Generally speaking, the higher the percentage of the chitosan used, the longer the bioabsorption time. Therefore, achieving optimum or near optimum performance of the hemostatic gauze calls for a balance of various ingredients.

Xanthan is a synthetic, water-soluble biopolymer typically made by fermentation of carbohydrates. Solid materials formed from xanthan alone or galactomannan alone are typically highly soluble in water, which is an advantageous property for a bioabsorbable dressing. But such materials do not typically provide the structure needed for the wound dressing to persist for an adequate period of time. Thus, there is a need for bioabsorbable hemostatic compositions that are sturdy enough to withstand manual pressure and which are reasonably uncomplicated to use, especially in emergency situations such as life-threatening traumas wherein stemming blood flow as fast as possible can be critical.

The use of a mixture of etherized celluloses and polysaccharide gums (advantageously, two) pursuant to some embodiments of the present invention results in a material having a highly controllable solubility that can be adjusted for optimal or near-optimal properties for each wound dressing application. Solid dressings made with a mixture of etherized cellulose, chitosan, nonionic surfactants, xanthan gum and at least one galactomannan, such as guar gum or locust bean gum is found to have several advantageous properties including: substantially instant stoppage of bleeding, rapid healing, controllable solubility in body fluids, short bioabsorption time, substantially free of side-effects, hypoallergic, low risk of infection, high liquid absorbency, high speed of liquid absorption, durable but controllable consistency, biodegradability, and low cost.

Galactomannans are polysaccharides containing both galactose and mannose residues. Advantageously, the galactomannans are selected from the group consisting of guar gums (wherein the galactose to mannose ratio is about 1:2), locust bean gums (wherein the galactose to mannose ratio is about 1:4) and mixtures thereof.

The water-soluble polysaccharide hydrocolloids used in compositions pursuant to some embodiments of the present invention are advantageously selected from the group consisting of glucosaminoglycans and some naturally occurring gums. Especially advantageous glucosaminoglycans for use in some embodiments of the present invention include guar gum and locus bean gum. The naturally occurring gums include xanthan gum and gum Arabic. A particularly advantageous usage level of the polysaccharide gums in some embodiments of the present invention is in the range from about 15% to about 30%. A particularly advantageous ratio of etherized cellulose to glucosaminoglycans and naturally occurring gums is in the range from about 10:3:2 to about 10:2:1.

Important advantages of using the combination of etherized polymers and polysaccharide gums pursuant to some embodiments of the present invention include: (1) the synergistic effect on the solubility in body fluids obtainable by varying the type and ratio of etherized polymers and polysaccharide gums used. For example, gauze made of etherized hydroxy propyl cellulose, methyl hydroxy propyl cellulose, and methyl hydroxy ethyl cellulose in the ratio of about 2:1.5:1.25 can be essentially completely absorbed by the body in about 4 hours. However, if hydroxy propyl cellulose, methyl hydroxy propyl cellulose, methyl hydroxy ethyl cellulose, guar gum, and xanthan gum are used in the ratio of about 2:1.5:1.25:1.00:1.15, the body absorption time decreases to about 2 hours. (2) Varying the type and ratio of etherized polymers and polysaccharide gums used can increase versatility for different applications and can allow the custom tailoring of the hemostatic gauze with specific absorption times as might prove advantageous for different treatment applications. Advantageously, the dispersion is in a solution or gel, and the solvent is an aqueous solvent. More advantageously, the solvent consists essentially of water. Advantageously, the total weight concentration of the xanthan and the galactomannans in the dispersion pursuant to some embodiments of the present invention is in the range from about 2 mg/ml (milligram/milliliter) to about 20 mg/ml. The dispersion will typically be a transparent aqueous gel.

The nonionic surfactant ingredient used in some compositions pursuant to some embodiments of the present invention is advantageously selected from the group consisting of saturated and/or unsaturated primary, secondary, and/or branched, amine, amide, amine-oxide, fatty alcohols, fatty acids, alkyl phenols, and/or alkyl aryl carboxylic acids and/or ester compounds, each having typically from about 6 to about 22 carboxyl groups in an alkyl or alkylene chain, wherein at least one active hydrogen of said compound is ethoxylated with about 30 ethylene oxide moieties to provide an HLB (Hydrophile-Lipophile Balance) from about 8 to about 20.

The surfactants used pursuant to some embodiments of the present invention may be one or more nonionic surfactants. When a combination of surfactants is used, the first component may be a fatty alcohol, while the second component may be any other primary, secondary, and/or branched, amine, amide, amine-oxide, fatty acid, alkyl phenol, and/or alkyl aryl carboxylic acid and/or ester compound, advantageously each having from about 10 to about 18 carboxyl group in an alkyl or alkylene chain, wherein at least one active hydrogen of said compound is ethoxylated with about 30 ethylene oxide moieties to provide an HLB in the range from about 12 to about 18.

In order to achieve the desired property of instant or very rapid wetability, the usage level of chitosan should be in the range from about 2% to about 15%, the ratio between the first and second component of the binary surfactant mixture should be kept within about 1:8 and 1:2, advantageously between about 1:5 and about 1:3. The total concentration of surfactants that is desirable in the final product depends on the properties of the other ingredients, but usually is advantageously in the range from about 0.1% to about 3% (w/w), but more advantageously lies in the approximate range of between 0.1% and 1.5% (w/w).

Fatty alcohols are typically used to achieve the desired level of softness of the product. Examples of fatty alcohols include glycerol, polyethylene glycol, propylene glycol, glycerol monoesters with fatty acids or other fatty alcohols typically used pharmaceutically. The concentration of the fatty alcohol in the product usually ranges between about 0.1% and about 1.5% (w/w).

The raw materials used in making etherized celluloses pursuant to some embodiments of the present invention include a source of cellulose, typically cotton, defatted cotton, recycled cellulose, sponges, fibrillated wood pulp, among others.

The methods of making etherized celluloses pursuant to some embodiments of the present invention include the steps of placing the raw materials in a closed chemical reactor and adding alkaline metal hydroxide. The substances used are advantageously aqueous sodium hydroxide, aqueous potassium hydroxide. Also, halogenated alkyl compounds, such as methyl chloride, ethyl chloride, and propyl chloride, among others, as well as chloroacetic acid, chloropropanoic acid and chlorobutanoic acid, among others may be employed. Additional alkenyl oxides may also be used, such as ethylene oxide and propylene oxide, among others. The mixture is heated at a temperature from about 30 deg. C. to about 160 deg. C. for about 1-6 hours. More advantageous heating parameters are found to be from about 80 deg. C. to about 150 deg. C. for about 1-2 hours, or from about 60 deg. C. to about 150 deg. C. for about 1-3 hours.

The product is then neutralized with C1-C5 lower alkyl alcohols which include methanol, ethanol, propanol, butanol, pentanol, and isopropyl alcohol, together with acids such as acetic acid or phosphoric acid, to a pH of about 4.5-8. The resulting product is then washed with approximately 70%-90% ethanol until the halogen content in the product is lower than about 1%. Finally, the product is freeze-dried and pulverized. The etherized cellulose pursuant to some embodiments of the present invention is selected from the group consisting of hydroxy propyl cellulose (with DS in the range from about 0.3 to about 2.5), methyl hydroxy propyl cellulose with DS about 0.6-2.8, and methyl hydroxy ethyl cellulose with DS about 0.5-2.6.

The method of making a bioabsorbable water-soluble, hemostatic gauze matrix pursuant to some embodiments of the present invention includes the steps of mixing one or more of the etherized cellulose compounds, (typically produced as described elsewhere herein), and a hemostatic compound in a non-aqueous solvent to form a fibrous pulp, said hemostatic compound typically comprising chitosan, one or more water-soluble polysaccharide gums, and one or more surfactants.

The non-aqueous solvent described above is advantageously selected from the group consisting of straight-chain or branched C1-C5 alcohols, ketones, aliphatic ethers, cycloaliphatic ethers, esters, nitrites, and aliphatic halogenated hydrocarbons. Advantageously, the solvent used is the alcohol of 95% to 100% ethanol. In some embodiments of the present invention, high shear mixing is used to produce substantially even dispersion of the material. The fibrous pulp is collected on forming fabric. “Forming fabric” denotes a material typically used during paper manufacturing that permits the drainage of the pulp solution while retaining the fibers. It provides mechanical support, imparts surface characteristics during pressing and drying, and is then released from the dried paper product. The forming fabric can be a variety of materials including, but not limited to, a Teflon or stainless steel mesh screen, and advantageously is a polyester woven fabric. In other embodiments, the fibrous pulp is collected onto the forming fabric under vacuum conditions. The wet pulp collected is subjected to heat compression and freeze dried. The product is first frozen in the temperature range from about −30 deg. C. to about −50 deg. C. for about 15-40 minutes, then freeze-dried. The normal cycle is about −30 deg. C. to about +25 deg. C. overnight to produce the sponge.

The fibrous pulp is then subjected to a paper-making process to form a paper product. The paper-making process includes the steps of first separating the fibrous pulp from a non-aqueous solvent and collecting it onto a forming fabric which include stainless steel mesh, polyester fabric, Teflon mesh, among others, then treating it with heat compression. The pulp is then subject to vacuum for defoaming purposes. The resulting product is then pressed and freeze-dried into the form of a sponge.

In some embodiments of the present invention, the method involves precipitating the components of the hemostatic composition either separately or together in a non-aqueous solvent, admixing the precipitated components under conditions sufficient to form a fibrous pulp, and then collecting, pressing and drying the fibrous pulp to produce a solid, bioabsorbable hemostatic composition. The materials according to some embodiments of the present invention may be in the form of a sponge. The sponge material according to some embodiments of the present invention may be provided in any shape, but is advantageously provided as a wound dressing layer having a thickness from about 1 mm to about 5 mm. Advantageously, the sponge material has a water absorbency of at least approximately 30 g/g.

Some embodiments of the present invention use the hemostatic composition for topical treatment to stop bleeding of wounds due to trauma, surgery or other causes. In addition, the methods pursuant to some embodiments of the present invention include hemostatic composition(s) to inhibit or stop bleeding of an organ, such as the liver, kidney, spleen, pancreas or lungs, among others. In addition, the methods of some embodiments include inhibiting or stopping bleeding or fluid loss during surgery including, but not limited to, abdominal, vascular, urological, gynecological, thyroidal, neurosurgery, tissue transplant, and dental surgery. Some embodiments of the present invention relate to a method of rapidly stopping blood loss from a wound by applying to the wound a solid hemostatic composition containing a bioabsorbable polymer and other components which can provide advantageous wound-healing benefits.

Hemostatic compositions pursuant to some embodiments of the present invention are advantageously maintained in contact with a wound by applying light pressure for a period of time sufficient to arrest the blood and for blood clotting to occur. Generally, the hemostatic composition is maintained in contact with the wound surface for a period of about 20 seconds to about 10 minutes, advantageously about 20 seconds to about 5 minutes, and more advantageously about 20 seconds to about 2 minutes. Some embodiments of the present invention can also include an elastic bandage which can be wrapped around the patch so as to provide pressure to the wound site.

Hemostatic compositions pursuant to some embodiments of the present invention can also be made into an attachment to an adhesive tape, or adhered to an adhesive backing in a BAND-AID form. The type of adhesive used can be any type of medically acceptable adhesive. The adhesive used is advantageously a porous type which can allow air diffusion to the surface that is in contact with the wound. Various forms, shapes, sizes and types of hemostatic bandage can be made to fit various needs, such as waterproof, individual sterile package, boxed including various shapes and sizes of the bandage, or in a kit designed for emergency or military use that can also contain disposable pre-sterilized instruments, such as scissors, scalpel, clamp, tourniquet, elastic or inelastic bandages, among others.

Another advantage of some embodiments of the present invention relates to ease of use. Typically, no specialized training is needed. Use in the field is also quite feasible, such as in trauma packs for soldiers, rescue workers, ambulance/paramedic teams, firemen, and by emergency room personnel, and in first aid kits for use by the general public. Thus, utilization of the hemostatic compositions of some embodiments of the present invention is expected to result in a reduction of fatalities due to trauma and, by more effectively stopping bleeding, can reduce the drains on the supply of stored blood, which can be in serious shortage during a disaster situation.

Pharmaceutically active ingredients and therapeutic agents which exhibit absorption problems due to solubility limitations, degradation in the gastro-intestinal tract, or extensive metabolism, are well suited to be used in hemostatic compositions pursuant to some embodiments of the present invention. Examples of such therapeutic agents include hypnotics, sedatives, antiepileptics, awakening agents, psychoneurotropic agents, neuromuscular blocking agents, antispasmodic agents, antihistaminics, antiallergics, cardiotonics, antiarrhythmics, diuretics, hypotensives, vasopressors, antitussive expectorants, thyroid hormones, sexual hormones, antidiabetics, antitumor agents, antibiotics, chemotherapeutics, and narcotics, among others.

The amount of drug to be incorporated into the hemostatic composition depends on the type of drug and its intended effect on the patient. Typical concentrations of drug in hemostatic agent is between about 0.01% and 15% (w/w), but can be higher if necessary to achieve the desired effect.

Flavorings (which include breath freshening compounds like menthol, peppermint oils, spearmint oils, among others), and/or other agents used for dental and/or oral cleansing (such quarternary ammonium bases) may be incorporated into hemostatic composition pursuant to some embodiments of the present invention. Flavor enhancers like tartaric acid, citric acid, vanillin, or the like may also be used. FD&C (Food, Drug & Cosmetic Act) colorants which may optionally be mixed in the hemostatic compositions must be safe in terms of toxicity and should be accepted by the Food and Drug Administration for such use.

Specific procedures for making hemostatic compounds, formulations and structures pursuant to some embodiments of the present invention are presented. Such procedures are illustrative and not limiting as different procedures deriving from, and/or related to, the techniques presented herein will be apparent to those having ordinary skills in the art and are included within the scope of the present invention.

### EXAMPLE 1

Place 50 g defatted cotton in a closed chemical reactor. Add 750 ml 50% w/w sodium hydroxide aqueous solution. Allow the reaction to proceed under constant agitation at room temperature for 2 hours. Then add about 150 ml of 50% w/w chloroacetic acid, 200 ml of ethylene oxide and 400 ml of propylene oxide to the solution for continued reaction for 8 hours. The mixtures are then heated to 50 deg. C. to 55 deg. C. and maintained at the elevated temperature for 5 hours while mildly agitating the slurries. The derivatized fibers are recovered by filtration. The resulting product is neutralized with reagent grade acetic acid (84%) to a pH of about 7.0. The recovered fibers are then slurried in about 250 ml of 100% isopropanol. The fibers recovered from the final washing are slurried in media containing lower proportions of the organic solvents to form slurries of about 10% consistency. Following pressing to expel excess liquid, the mats or sheets were freeze-dried. The final product is hydroxyl propyl cellulose with DS of 1.2 to 1.4. The finished product is then sterilized and packaged. Sterilization is advantageously accomplished by irradiation with gamma rays from a cobalt-60 source, or other sterilization methods known in the art.

50 grams of a mixture containing 75% by weight of the above freeze-dried hydroxyl propyl cellulose, 5% by weight of 90% deacetylated, decrystallized chitosan (supplied by Indian Sea Foods), 12% by weight of gum Arabic (supplied by Gum Technology Corp.), 7% of locus bean gum (supplied by Danisco Inc.), 0.4% by weight of glycerol and 0.1% by weight of Polysorbate 80 (polyoxyethylene sorbitan monooleate, (x)-sorbitan mono-9-octadecenoate poly(oxy-1,2-ethanediyl) are added to 1,000 ml 95% ethanol and mixed at high shear in a Virishear 1700 homogenizer at 5,000 rpm for 50 seconds, then the resulting pulp solution is then diluted with 250 ml of 95% ethanol. The sample is collected on forming fabric using a Millipore filter housing (dia.=7.4 cm). The wet sample is pressed at 2 metric tons for 20 seconds, frozen to −40C, and freeze dried into a sponge, then packaged and sterilized. Typically, the product is packaged and the package and product is sterilized as a unit. However, this is not an inherent limitation, and any procedure resulting in a sterilized product packaged so as to retain sterility can be used.

### EXAMPLE 2

Place 50 g cotton in a closed chemical reactor. Add 700 ml 50% w/w potassium hydroxide aqueous solution. Allow the reaction to proceed under constant agitation at room temperature for 2 hours. Then add about 100 ml of 50% w/w, chloroacetic acid, 50 ml of chloropropanoic acid, 400 ml of ethylene oxide and 200 ml of propylene oxide to the solution for continued reaction for 8 hours. The resulting product is neutralized with reagent grade acetic acid (84%) to a pH of about 6.0. Then 70-90% ethanol is used to wash the finished product until the chlorine content in the product is lower than 1%. The final product is methyl hydroxyl propyl cellulose with DS of 1.0 to 1.2. The finished product is then freeze dried, packaged and sterilized.

50 grams of a mixture containing 75% by weight of the above freeze-dried methyl hydroxyl propyl cellulose, 5% by weight of 90% deacetylated, decrystallized chitosan, 12% by weight of gum Arabic, 7% of guar gum, 0.4% by weight of propylene glycol and 0.1% by weight of Polysorbate 80 (polyoxyethylene sorbitan monooleate, (x)-sorbitan mono-9-octadecenoate poly(oxy-1,2-ethanediyl, supplied by are added to 1,000 ml 95% ethanol and mixed at high shear in a Virishear 1700 homogenizer at 4,500 rpm for 30 seconds. Then the resulting pulp solution is diluted with 250 ml of 95% ethanol. The sample is collected on forming fabric using a Millipore filter housing (dia.=7.4 cm). The wet sample is pressed at 2 metric tons for 30 seconds, frozen to −40C, and freeze-dried into a sponge, then packaged and sterilized.

### EXAMPLE 3

Place 10 g recycled cellulose in a closed chemical reactor. Add 150 ml 50% w/w sodium hydroxide aqueous solution. Allow the reaction to proceed under constant agitation at room temperature for about 1 hour. Then about 30 ml of 50% w/w chloroacetic acid, 65 ml of ethylene oxide and 160 ml of propylene oxide are added to the solution for continued reaction for 8 hours. The resulting product is neutralized with reagent grade acetic acid (84%) to a pH about 6.2. Then 70-90% isopropyl alcohol is used to wash the finished product until the chlorine content in the product is lower than 1%. The final product is hydroxyl propyl cellulose with DS of 1.2 to 1.4. The finished product is then freeze-dried, packaged and sterilized.

50 grams of a mixture containing 75% by weight of the above freeze-dried hydroxyl propyl cellulose, 5% by weight of 90% deacetylated, decrystallized chitosan, 8% by weight of xanthan gum, 11% of locus bean gum, 0.4% by weight of propylene glycol and 0.1% by weight of 1.5 g Kollidon 30 (Polyvinylpyrrolidone, povidone, supplier: BASF), are added to 1,000 ml 95% ethanol and mixed at high shear in a Virishear 1700 homogenizer at 5,000 rpm for 50 seconds. Then the resulting pulp solution is diluted with 250 ml of 95% ethanol. The sample is collected on forming fabric using a Millipore filter housing (dia.=7.4 cm). The wet sample is pressed at 2 metric tons for 20 seconds, frozen to −40C, and freeze dried into a sponge, then packaged and sterilized.

Although various embodiments which incorporate the teachings of the present invention have been shown and described in detail herein, those skilled in the art can readily devise many other varied embodiments that still incorporate these teachings.

What is claimed is:

1. A hemostatic composition comprising:
one or more species of chitosan; and,
one or more species of etherized cellulose; and,
one or more species of nonionic surfactant; and,
one or more water-soluble species selected from the group consisting of polysaccharide hydrocolloids, polysaccharide gums and mixtures thereof.

2. A composition as in claim 1 wherein said one or more species of etherized cellulose is selected from the group consisting of: hydroxy propyl cellulose, methyl hydroxy cellulose, methyl hydroxy ethyl cellulose, and mixtures thereof.

3. A composition as in claim 2 wherein said one or more species of etherized cellulose is a mixture of hydroxy propyl cellulose, methyl hydroxy cellulose and methyl hydroxy ethyl cellulose in the approximate proportions of 1:2:1.5.

4. A composition as in claim 2 wherein:
said hydroxy propyl cellulose has a DS in the range from about 0.3 to about 2.5; and,
said methyl hydroxy propyl cellulose has a DS in the range from about 0.6 to about 2.8; and,
said methyl hydroxy ethyl cellulose has a DS in the range from about 0.5 to about 2.6.

5. A composition as in claim 2 wherein:
said hydroxy propyl cellulose has a DS in the range from about 0.8 to about 2.0; and,
said methyl hydroxy propyl cellulose has a DS in the range from about 1.2 to about 2.5; and,
said methyl hydroxy ethyl cellulose has a DS in the range from about 1.0 to about 2.2.

6. A composition as in claim 1 wherein said one or more species of chitosan is selected from the group consisting of approximately 85% to approximately 90% deacetylated decrystallized chitosan and mixtures thereof.

7. A composition as in claim 1 wherein said one or more species of chitosan is present in an amount in the range from about 2% to about 15% by weight.

8. A composition as in claim 6 wherein said one or more species of etherized cellulose, and said one or more species of chitosan are present in a ratio in the range from about 10:1 to about 15:1.

9. A composition as in claim 1 wherein said one or more water soluble species comprises xanthan gum and at least one galactomannan.

10. A composition as in claim 9 wherein said at least one galactomannan is selected from the group consisting of gar gums, locust bean gums, and mixtures thereof.

11. A composition as in claim 10 wherein said gar gums have a galactose to mannose ratio of about 1:2, and wherein said locust bean gums have a galactose to mannose ratio of about 1:4.

12. A composition as in claim 1 wherein said one or more water-soluble species comprises one or more species of glucosaminoglycan and one or more species of naturally occurring gums selected from the group consisting of xanthan gum, gum Arabic and mixtures thereof.

13. A composition as in claim 12 wherein said one or more species of glucosaminoglycan is selected from the group consisting of gar gums, locus bean gums and mixtures thereof.

14. A composition as in claim 1 wherein said one or more water-soluble species is present in an amount in the range from about 15% to about 30% by weight.

15. A composition as in claim 12 wherein said one or more species of etherized cellulose, said one or more species of glucosaminoglycan and said one or more species of naturally occurring gums are present in a ratio in the range from about 10:3:2 to about 10:2:1 by weight.

16. A composition as in claim 1 wherein said one or more species of nonionic surfactant is selected from the group consisting of: saturated or unsaturated, primary, secondary or branched amine, amide, amine-oxide, fatty alcohols alkyl phenols, alkyl aryl carboxylic acids, esters and mixtures thereof; wherein each of said one or more species of nonionic surfactant has from about 6 to about 22 carboxylic groups in an alkyl or alkylene chain, and is sufficiently ethoxylated so as to provide an HLB in the range from about 8 to about 20.

17. A composition as in claim 1 wherein said one or more species of nonionic surfactant is present in an amount in the range from about 0.1% to about 3% by weight.

18. A composition as in claim 1 wherein said one or more species of nonionic surfactant is present in an amount in the range from about 0.1% to about 1.5% by weight.

19. A composition as in claim 1 wherein:
said one or more species of etherized cellulose is present in an amount from about 55% to about 95% by weight; and,
said one or more species of chitosan is present in an amount from about 0.5% to about 15% by weight; and,
said one or more water-soluble species is present in an amount from about 5% to about 50% by weight; and,
said one or more species of nonionic surfactant is present in an amount from about 0.1% to about 5% by weight; and,
further comprising acetic acid present in an amount from about 0.01% to about 10% by weight.

20. A composition as in claim 19 wherein:
said one or more species of etherized cellulose is present in an amount from about 65% to about 85% by weight; and,
said one or more species of chitosan is present in an amount from about 1% to about 5% by weight; and,
said one or more species of water-soluble polysaccharide gum is present in an amount from about 15% to about 25% by weight; and,
said one or more species of nonionic surfactant is present in an amount from about 0.2% to about 2% by weight.

21. A method of making etherized cellulose comprising:
a) placing reactants in a closed chemical container and heating in the range from about 30 deg. C. to about 160 deg. C. for a time from about 1 hour to about 6 hours, producing thereby a first product; and
b) treating said first product with acid to produce a second product having a pH in the range from about 4.5 to about 8; and,
c) washing said second product with approximately 70% to 90% ethanol until the halogen content is lower than about 1%, producing thereby a third produce; and,
d) freeze drying and pulverizing said third product;
wherein said reactants comprise a source for cellulose, one or more species of alkaline metal hydroxide, one or more species of halogenated alkyl compounds and optionally one or more species of alkenyl oxides.

22. A method of making a hemostatic gauze comprising:
mixing one or more species of etherized cellulose with a hemostatic compound in a non-aqueous solvent forming thereby a fibrous pulp; and,
separating said fibrous pulp from said non-aqueous solvent and collecting said fibrous pulp on a forming fabric; and,
pressing said collected fibrous pulp and freeze drying;
wherein said hemostatic compound comprises one or more species of chitosan, one or more species of water-soluble polysaccharide gum, and one or more species of surfactant.
